(19) United States
(12) Patent Application Publication
(10) Pub. No.: US 2004/0058865 A1
(43) Pub. Date: Mar. 25, 2004

(54) Homing peptide multimers, their preparation and uses

(76) Inventors: Samuel J Danishefsky, Engelwood, NJ (US); Lawrence C. Fritz, Rancho Santa Fe, CA (US)

Correspondence Address: BIOTECHNOLOGY LAW GROUP, 658 MARSOLAN AVENUE, SOLANA BEACH, CA, 92075, US

(21) Appl. No.: 10/433,212

(22) PCT Filed: Nov. 26, 2001
(86) PCT No.: PCT/US01/44154

(51) Int. Cl. A61K 38/17; C07K 14/47

(52) U.S. Cl. 514/12; 530/324

(57) ABSTRACT

Synthetic multimeric ligands that provide for enhanced cell-, and organ-specific targeting are described and claimed, as are methods of their preparation and use

### FIELD OF THE INVENTION

The present invention concerns synthetic multimeric ligands that provide for enhanced cell-, tissue-, and organ-specific targeting.

### BACKGROUND OF THE INVENTION

The following description of the background of the invention is provided to aid in understanding the invention, but is not admitted to be or to describe prior art to the invention.

The targeted delivery of drugs, prodrugs, or other therapeutic agents to the cells where they are needed may improve pharmacological treatment of diseases. Targeted delivery can shorten drug delivery and or response time and also lower effective dosages of drugs, thus reducing undesired side effects which arise from elevated dosage levels.

Tumors present a therapeutic challenge, especially those which are difficult to surgically excise or to treat with radiation, particularly if they are difficult to locate, metastatic and/or are close to tissues that are critical for the well-being of the patient. Also, some tumors cannot be effectively treated by standard chemotherapies since the fraction of administered therapeutic agent that will reach the tumor is very small. The effective dosage cannot be increased by simply administering higher dosages to a patient, since elevated dosage levels may lead to unacceptable side effects. Moreover, effective dosage levels may vary from patient to patient, and the dosage level which brings on deleterious side effects may vary from patient to patient. In addition, conventional drug therapies directed towards tumors are targeted against processes such as cell growth or division that occur in both normal and cancerous cells, resulting in pronounced toxicity to normal cells, tissues, and organs.

Several attempts have been disclosed that concern targeted delivery of chemical agents. For example, U.S. Pat. No. 5,639,737 discloses that growth or metastasis of malignant tumors associated with Hodgkin's disease can be inhibited by administering lactose to a patient. When lactose was conjugated to a cytotoxic substance and this conjugate was administered to a patient, both treatment of the tumor and prevention of metastasis were reportedly observed.

U.S. Pat. No. 5,490,988 discloses that antibodies which bind to target sites via antibody/antigen binding may be extended by the addition of an additional peptide; such a peptide “extension” is then a useful “handle” for the attachment of a therapeutic agent. U.S. Pat. No. 5,455,027 discloses that peptides may be copolymerized with water soluble polyethylene glycol (PEG) linkers and that such “copolymers” may be useful for drug delivery. U.S. Pat. No. 5,747,646 discloses the liking of polyethylene glycol conjugates to one or more amino groups of the proteins. U.S. Pat. No. 5,482,996 discloses a method of preparing water-insoluble, protein-containing polymers and also a method of incorporating biologically active proteins into water-insoluble polymers.

### SUMMARY OF THE INVENTION

A homing peptide is any peptide that provides for cell-, tissue-, or organ-specific targeting. A homing peptide multimer is a molecule comprising more than one homing peptide; such molecules allow the simultaneous interaction of more than one peptide with another biological entity, such as a peptide receptor molecule, or cell surface antigen or epitope. In principle, the strength of such multiple interactions is much stronger than the interaction between a single peptide and a corresponding single receptor.

Any suitable approach can be used to prepare homing peptide multimers of the invention While some embodiments concern the more or less random conjugation of peptide(s) and linker(s), and represent a desirable way to rapidly generate libraries of homing peptide multimers, a preferred embodiment of the present invention concerns the ability to control the location(s) and nature of the conjugation between homing peptide(s) and linker(s). Because the linking of a peptide to a linker or scaffold may compromise the functional integrity of a homing peptide (especially those portions of a homing peptide closest to the linker or scaffold), it is important to minimize or control this factor.

Two preferred embodiments of homing peptide multimers according to the invention are shown in FIG. 1 and are referred to hereinafter as “serial” and “parallel” homing peptide multimers. Numerous combinations of these two basic designs may also be envisioned, for example, linked “serial” and “parallel” homing peptide multimers, or serial and parallel homing peptide multimers having branched scaffolds or linkers. Such homing peptide multimers fall within the scope of molecules comprising more than one horning peptide as they allow the simultaneous interaction of more than one horning peptide with another biological entity, such as a targeted molecule, cell, tissue, or organ.

In the serial approach, peptides are linked via intervening linkers (“linker” means any bond, e.g., a covalent bond, an ionic bond, and a hydrogen bond, atom, group of atoms, molecule, or group of molecules disposed between two molecules linked by the linker). “Peptide” means any synthetic or naturally occurring sequence of amino acid residues linked by peptide bonds. “Amino acid residue” refers to a residue of the amino acid after incorporation into a peptide, which incorporation results in the loss of one or more atoms from the amino acid. “Amino acid” refers to any synthetic or naturally occurring molecule comprising an amino group and a carboxylic acid group. Preferred amino acids are α-amino carboxylic acids, particularly those that are incorporated into proteins in nature. Peptides may be linked “end-to-end” (via each peptide's C or N-terminus), “end-to-sidechain,” via reactive functional groups present on residues within a peptide sequence, or “side-chain-to-side chain”, via reactive functional groups present on residues within a peptide sequence. Methods to precisely control where and how linkers join peptides are a preferred aspect of the present invention and are discussed furer in the detailed description of the invention.

In the parallel approach (FIG. 1, right hand side), ends or side chain(s) of homing peptide are joined to a scaffold (a “scaffold” is any molecule that provides a molecular framework for an array of other molecules linked thereto). Just as in the serial approach, either end (C or N-terminus) of a homing peptide can be coupled to the scaffold and novel methods to selectively link either end (C or N-terminus) to a scaffold are a preferred aspect of the present invention and are discussed in the detailed description of the invention.

The peptide multimers disclosed herein are primarily intended be used as homing peptides for the targeting of tumors, but may be administered as therapeutic agents alone or in combination with drugs or prodrugs which are effective against a disease or condition. A therapeutic agent, e.g., a drug or prodrug, is any compound or formulation thereof which is effective in helping to prevent or treat a disease or condition. “Effective in helping to prevent or treat a disease or condition” indicates that administration in a clinically appropriate manner results in a beneficial effect for at least a statistically significant fraction of patients, such as a improvement of symptoms, a cure, a reduction in disease load, reduction in tumor mass or cell numbers, extension of life, improvement in quality of life, or other effect generally recognized as positive by medical doctors familiar with treating the particular type of disease or condition.

In yet another aspect of the invention, drug molecules, prodrug molecules, or other therapeutic agents may be linked to homing peptide multimers via covalent bonds or non-covalent bonds, e.g., ionic, electrostatic, van der Waals bonds. In this way, homing peptide multimers serve as “molecular homing devices” for the targeting of drugs or other therapeutic agents to specific cells, tissue, or organs. A release mechanism for the drug or prodrug which coincides with the arrival of the drug or prodrug at the targeted cell may be triggered by local conditions at the diseased organ, tissue, or cells, e.g, the reversible reductive cleavage of a disulfide bond. The pendent drug or prodrug, whether released or not, acts as a therapeutic agent at the target site.

### BRIEF DESCRIPTION OF THE FIGURES

FIG. 1 illustrates two embodiments of peptide multimers according to the invention.

FIG. 2 illustrates a commonly accepted mechanism for the coupling of carbon electrophiles and carbon nucleophiles to generate a new carbon-carbon bond in the presence of a transition metal catalyst. FIG. 2 is intended for illustrative purposes only, and the methods disclosed in the present invention are in no way limited by FIG. 2.

### DETAILED DESCRIPTION OF THE INVENTION

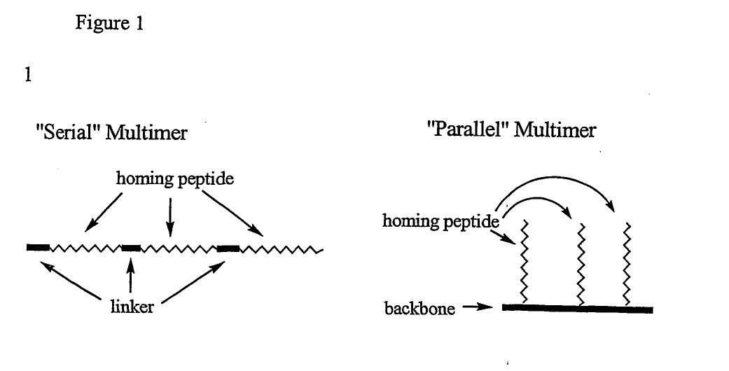

One general approach to the synthesis of homing peptide multimers is depicted in FIG. 1, right-hand side) and designated a parallel approach. Scaffold molecules are molecules that provide a molecular framework for an array of other molecules linked thereto. Preferred scaffolds include linear, but also branched molecules that provide a plurality of functional groups suitable for coupling to homing peptides. Examples of preferred scaffolds include, but are not limited to molecules having a linear or branched backbone chain substituted with functional groups which may readily link other functional groups attached to homing peptides. Specific examples of such scaffolds include peptides, diols (glycols), amino alcohols, diamines, glycerols, polyamines, pentoses, and hexoses, and their mixed amino analogs as well as “starburst” dendrimers. Other preferred scaffolds include linear and branched molecules derived from the oligomerization or polymerization of epoxides, aziridines and other strained-ring monomers, also comprising substituted norbomene, substituted 7-oxanorbomene, or related strained cyclic monomers characteristically used in ring-opening metathesis polymerization (ROMP).

In a preferred embodiment of the present invention, functional groups on a scaffold lsuitable for coupling to proteins may be carbon nucleophiles or carbon electrophiles. The terms “nucleophile” and “electrophile” have their usual meanings familiar to synthetic and/or physical organic chemistry. Carbon electrophiles typically comprise one or more alkyl, alkenyl, alkynyl or aromatic (sp³, sp², or sp³ hybridized) carbon atom substituted with any atom or group having a Pauling electronegativity greater than that of carbon itself. Examples of preferred carbon electrophiles include but are not limited to carbonyls (especially aldehydes and ketones), oximes, hydrazones, epoxides, aziridines, alkyl-, alkenyl-, and aryl halides, acyls, sulfonates (aryl, alkyl and the like). Other examples of carbon electrophiles include unsaturated carbons electronically conjugated with electron-withdrawing groups, examples being the β-carbon in α,β-unsaturated ketones or carbon atoms in fluorine substituted aryl groups. In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Preferred carbon electrophiles are those compatible with water or other polar solvents used to facilitate reactions of proteins, and include carbonyls, epoxides, aziridines, cyclic sulfates and sulfamidates, and alkyl vinyl and aryl halides. Methods of generating such carbon electrophiles, especially in ways which yield precisely controlled products, are well known to those skilled in the art of organic synthesis.

Suitable carbon nucleophiles include, but are not limited to alkyl, alkenyl, aryl and alkynyl Grignard, organolithium, organozinc, and related organometallic reagents. Most preferred organometallic carbon nucleophiles include but are not limited to alkyl-, alkenyl-aryl- and alkynyl-tin reagents (organostannanes), alkyl-, alkenyl-, aryl- and alkynyl borane reagents (organoboranes and organoboronates); these carbon nucleophiles have the advantage of being kinetically stable in water or polar organic solvents, the preferred solvents for protein chemistry. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with preferred carbon electrophiles, engender new carbon-carbon bonds between the carbon nucleophile and carbon electrophile.

Other preferred nucleophiles suitable for coupling to carbon electrophiles include but are not limited to primary and secondary amines, thiols, thiolates, and thioethers, alcohols, alkoxides. These preferred nucleophiles, when used in conjunction with preferred carbon electrophiles, typically generate heteroatom linkages (C—X—C) between the homing peptides and scaffold, wherein X is a hetereoatom, e.g, oxygen or nitrogen.

Other methods suitable for selectively linking homing peptides to scaffolds utilize cycloaddition reactions. Like the nucleophile/electrophile linking methodology already described, these methods utilize complementary functional groups. Typically, cycloaddition reactions fuse unsaturated precursors and provide 5- and 6-membered ring products at the expense of one or more unsaturated (c) bond in the precursor. Examples of such reactions are 1,3-dipolar cycloadditions, Diels-Alder and hetero Diels-Alder cycloadditions. The products expected from the cycloaddition of complementary functional groups are highly predictable. Thus when such complementary functional groups are conjugated to homing peptides or scaffolds, one of ordinary skill in the art could selectively couple homing peptides to scaffolds and thereby construct homing peptide multimers having either the “parallel” or “series” structural motif illustrated in FIG. 1. These methods are fully intended to fall within the scope of the present invention and are incorporated by provident suggestion.

The intended targets of the homing peptide multimers disclosed herein are cancers or tumors of any type, including solid tumors and leukemias (including those in which cells are immortalized, including: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell, and transitional cell), histiocytic disorders, leukemia (e.g., b-cell, mixed-cell, null-cell, T-cell, T-cell chronic, HTLV-II-associated, lyphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, iemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's, experimental, Kaposi's, and mast-cell), neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia), and for treatment of other conditions in which cells have become immortalized. Other diseases intended to fall within the scope of those treatable by the compounds disclosed herein are listed in standard texts such as Cancer: Principles & Practice of Oncology, 5th edition by Vincent T. Devita, Steven A Rosenberg and Samuel Hellman (editors) Lippincott Williams & Wilkins, 1997; Harrison's Principles of Internal Medicine (14th Ed) by Anthony S. Fauci, Eugene Braunwald, Kurt J. Isselbacher, et al. (Editors), McGraw Hill, 1997, or Robbins Pathologic Basis of Disease (6th edition) by Ramzi S. Cotran, Vinay Kumar, Tucker Collins & Stanley L. Robbins, W B Saunders Co., 1998, or other texts described below, herein incorporated by reference.

Specific drugs which are effective in helping to prevent or treat a disease or condition are identified in the 1999 Physicians' Desk Reference (53rd edition), Medical Economics Data, 1998, or the 1995 United States Pharmacopeia XXIII National Formulary XVIII, Interpharm Press, 1994. Examples of antitumor therapeutic agents are known in the art and include but are not limited to, toxins, drugs, enzymes, cytokines, radionuclides; toxins include ricin A chain, mutant Pseudomonas exotoxins, diptheria toxoid, streptonigrin, boamycin, saporin, gelonin, and pokeweed antiviral proteins; antitumor therapeutic drugs and prodrugs drugs include but not limited to 5-fluorouracil (5-FU), daunorubicin, cisplatin, or cisplatinum, bleomycin, melphalan, taxol, tamoxifen, mitomycin-C, methotrexate, and ifosfamid. Radionuclides include radiometals. Photodynamic agents include porphyrins and their derivatives.

Prodrugs include chemical derivatives of a biologically-active parent compound which, upon administration, will eventually liberate the active parent compound in vivo. Use of prodrugs allows the artisan to modify the onset and/or duration of action in vivo. In addition, the use of prodrugs can modify the transportation, distribution or solubility of a drug in the body. Furthermore, prodrugs may reduce the toxicity and/or otherwise overcome difficulties encountered when administering pharmaceutical preparations

Pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutically acceptable compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such admiation.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol thie dosage unit may be determined by providing a valve to deliver a metered amount. Capsules and cartridges of e.g. gelatin for use in an inhaler or insuffator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or Iemulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble

A pharmaceutically acceptable carrier for any hydrophobic compound of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the peptide multimers compounds of the invention may be provided as pharmaceutically acceptable salts with pharmaceutically compatible counterions. Phannaceutically compatible salts may be formed with many acids, including but not limited to hydrochloric, sulfrric, acetic, lactic, tartaric, malic, succinic, etc. Salts tend to be more soluble in aqueous or other protic solvents The terms “formulation” and “liquid formulation” and the like are used herein to describe any pharmaceutically active drug by itself or with a pharmaceutically acceptable carrier. A formulation could be a powder, that may have previously been spray dried, lyophilized, milled, or the like, and may contain a large amount of inactive ingredients such as lactose or mannitol. The formulation is preferably in flowable liquid form having a viscosity and other characteristics such that the formulation can be aerosolized into particles which are inhaled into the lungs of a patient after the formulation is aerosolized, e.g. by being moved through a porous membrane. Such formulations are preferably solutions, e.g. aqueous solutions, ethanolic solutions, aqueous/ethanolic solutions, saline solutions, microcrystalline suspensions and colloidal suspensions. Formulations can be solutions or suspensions of drug in a low boiling point propellant or even dry powders. Dry powders tend to absorb moisture and the invention decreases the moisture content and makes it possible to deliver particles of powder which have a consistent size even when the surrounding humidity is variable.

The term “substantially dry” shall mean that the composition can include an amount of carrier (e.g. water or ethanol) which is comparable to (in weight) or less than the amount of drug in the particle. Preferably such particles consist essentially of only drug with no free carrier e.g., no free water, ethanol or other liquid that are the corresponding free base forms.

The following examples are offered by way of illustration and not by way of limitation.

### EXAMPLIS OF PREFERRED EMBODIMENTS

### Example 1

One novel way to construct homing peptide multimers relies on classical methodology: the polymerization N-carboxyanhydrides (oxazolidine-2,5-diones). These anhydrides, also known in the art as Leuch's anhydrides, are obtained by treating amino acids with phosgene, COCl₂, in aprotic solvents (Eq 1) [Bodanszky, in Peptide Chemistry: A Practical Textbook, 2^{nd} ed.; Springer Verlag: Berlin, 1993, pp. 136-137].

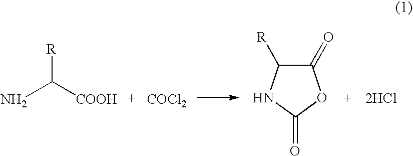

Herein, “amino acid” refers to any synthetic or naturally occurring molecule comprising an amino group and a carboxylic acid group. Preferred amino acids are α-amino carboxylic acids, particularly those that are incorporated into proteins in nature. “Peptide” means any synthetic or naturally occurring sequence of amino acid residues linked by peptide bonds.

Nucleophiles, including the amino moiety of an amino acid, readily cleave Leuch's anhydrides by attacking the electrophillic carbonyl carbon in the ring with concomitant formation of a new peptide bond (Eq 2):

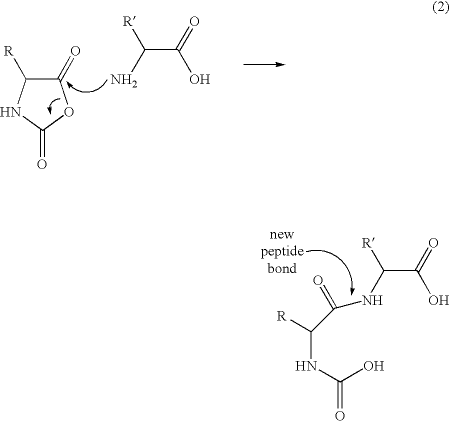

The carbamoic acid moiety of the coupling product is decarboxylated, regenerating a nucleophilic amine which ring-opens another equivalent of anhydride to generate trimeric and higher molecular weight peptides.

### Example 2

In another aspect of the present invention, homing peptide multirners are constructed by polymerizing amino acids having pre-formed peptide side chains (e.g., derivatized aspartic acid, X=carboxylate) using the classical Leuch's anhydride methodology (Eq 3).

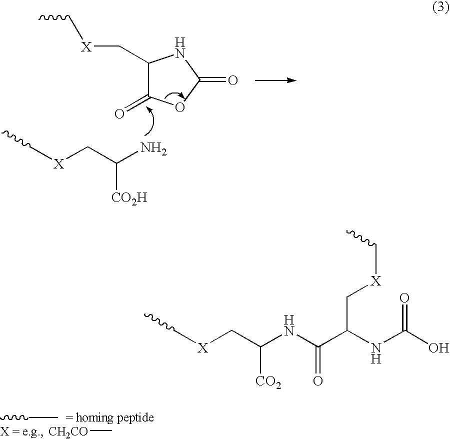

The carbamoic acid moiety of the dimeric peptide product in Eq 3 is decarboxylated to regenerate a free amino terminus which then ring-opens another equivalent of Leuch's anhydride to generate a “trimerc” homing peptide multimer (Eq 4):

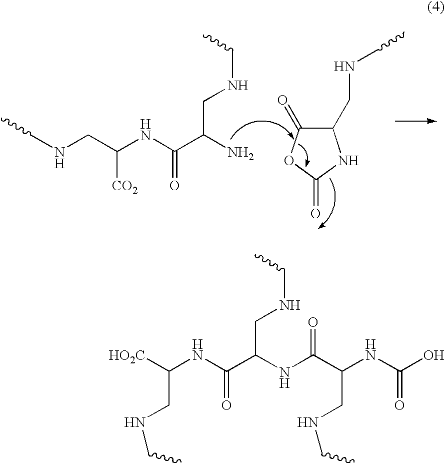

In one aspect of the invention, dimeric, trimeric and higher homing peptide multimers are prepared and used alone for therapeutic purposes (vide infra). In another aspect of the invention a free amino group of a peptide multimer acts as a nucleophillic coupling partner for the attachment of a drug or prodrug having a complementary electrophillic coupling partner (Eq 5):

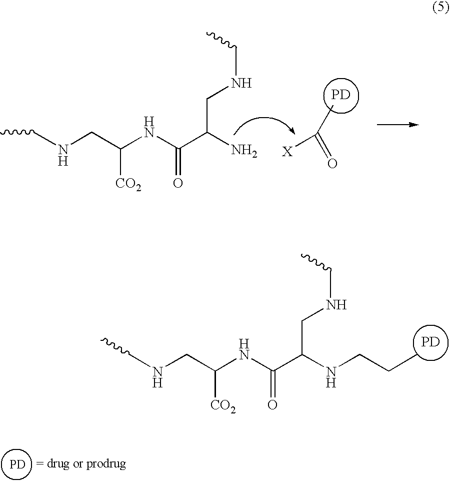

### Example 3

In another aspect of the present invention, hydrolytically stable macrocyclic trimers of amino acids are used as scaffold molecules (Eq 6). Such cyclic trimers have been reported [for example: J. Electroanal. Chem. Interfacial Electrochem. 1989, 266, 379-396]. Peptides having suitable reactive groups are appended to such cyclic trimers or larger ring analogs to give homing peptide multimers.

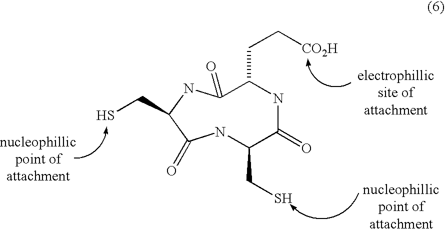

### Example 4

The ends of one, two, three or more homing peptides are tethered to simple di-, tri-, or poly-functional amines, or simple di-, tri-, or polyfunctional alcohols. In a preferred aspect of the invention, the arrangement of pendent homing peptides to the scaffold, e.g., which terminus (C or N) or the precise residue within a peptide sequence which makes a bond to the scaffold is controlled by the choice of coupling methodology. To illustrate, hydroxy groups of a scaffold molecule are transformed into carbon electrophiles using various methods well known to those skilled in the art of synthetic organic chemistry. Nucleophillic moieties at the ends or within a peptide sequence facilitate coupling of the homing peptide to the scaffold.

### Example 5

Scaffolds having hydroxy-or amine groups are converted into vinyl functional groups via reaction with an allylic electrophille, e.g., allyl bromide. Scaffolds having a plethora of vinyl groups are then epoxidized using methods known to those skilled in the art of organic synthesis. The result of such a synthetic sequence is an epoxide multimer useful for coupling an equivalent number of homing peptides, via a nucleophillic functional group attached to a homing peptide, Eq (7). A nucleophillic moiety within a homing peptide sequence could also facilitate coupling.

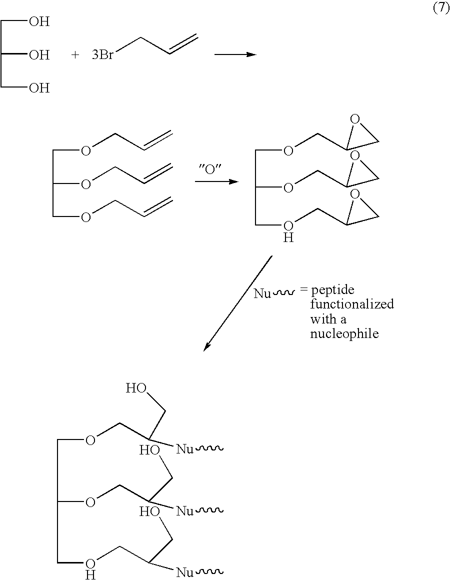

### Example 6

Scaffolds may be polymers, or repeating units of functionalized monomers Certain olefin polymerization reactions are highly efficient in that no by-products are formed during the linking of monomeric subunits and the chemistry is driven by the relief of ring-strain energies. One example of such a process is ring-opening metathesis polymerization, known in the art as (ROMP). ROMP produces hydrocarbon polymers of defined length from monomeric strained ring precursors. By appending a reactive functional group to each monomer unit, and subsequently attaching peptides to those functional groups, polymers having multiple appended peptides are produced (Eq 8).

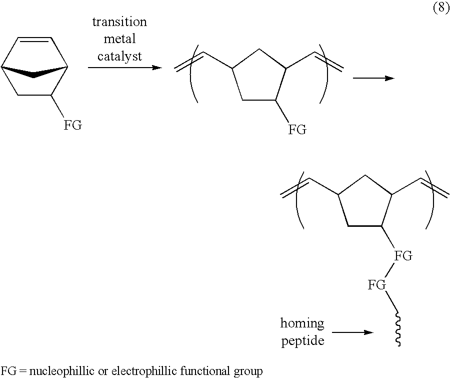

For additional information related to the ring-opening metathesis polymerization, see U.S. Pat. Nos. 6,121,236 and 6,083,708, U.S. Pat. No. 5,587,442 and Grubbs et al., J. Am. Chem. Soc. 2000, 122, 58-71.

### Example 7

In a preferred aspect of the present invention, one end of a homing peptide chain is conjugated to a functional group comprising a carbon nucleophile, most preferably an organoborane or boronate or organostannane moiety as previously described above; the termini of the scaffold molecule is thus transformed into a complementary carbon electrophile, most preferably, into a vinyl, aryl, or acetylenic halide, sulfonate, or acetate. Such carbon electrophiles and nucleophiles do not ordinarily react at any appreciable rate, but readily do so in the presence of a catalyst, for example, in the presence a low valent transition metal complexes, the most preferred transition metal complexes being palladium complexes wherein the palladium has a formal oxidation state of zero (0) or two (II). Other ligating groups associated with the tramrsition metal may also be present, e.g., phosphines, phosphonates, arsines, and other equivalents known to the art; these ligands serve chiefly to prevent the nucleation of Pd atoms into palladium metal. Co-catalysts such as CuI are also often present in such coupling reactions. For a general description of the coupling of carbon electrophiles and nucleophiles, see Comprehensive Organic Synthesis, Trost et al., Pergamon Press, Chapter 2.4: Coupling Reactions Between sp² and sp Carbon Centers, pp 521-549, and pp 950-953, hereby incorporated by reference.

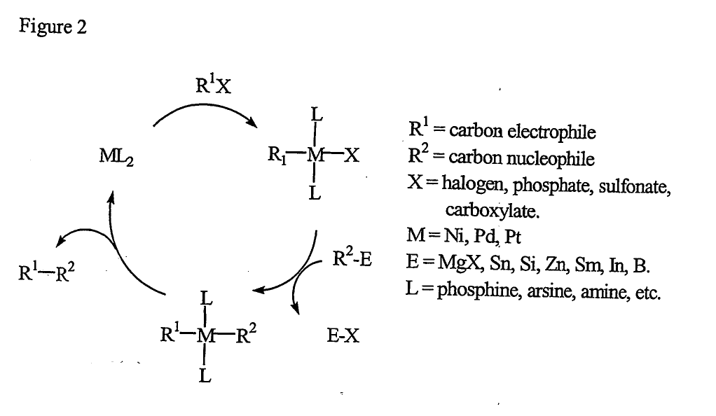

The carbon electrophile and carbon nucleophile generate a new carbon-carbon bond in the presence of a transition metal catalyst via a mechanism consistent with that outlined in FIG. 2.

The palladium-catalyzed coupling of organoboranes (E=B above) with carbon electrophiles to yields a new carbon-carbon bond and is known in the art as a Suzuki coupling [Suzuki et al. J. Am. Chem. Soc. 1989, 111, 314]. The palladium-catalyzed coupling of organostannane reagents (E=Sn in scheme above) and carbon electrophiles is known as a Stille coupling reaction [See Stille, J. K. Angew. Chem. Inlt. Ed. Engl. 1986, 25, 508 and Farina & Roth, Adv. Met.-Org. Chem. 1996, 5, 1-53]

In a preferred aspect of the present invention, the roles of a carbon electrophile and a carbon nucleophile can be interchanged; a carbon electrophile is attached to a homing peptide and a carbon nucleophile is attached to a scaffold. Transition metal-catalyzed coupling as described above will yield homing peptide multimers haying the opposite ends tethered to the scaffold; indeed one preferred aspect of the present invention is that functional groups used to conjugate homing peptides and scaffolds are modular in nature and are thus interchangeable. This preferred aspect of the invention also allows for the catenation of homing peptides using complementary synthetic carbon electrophiles and carbon nucleophiles in place of the natural components of a homing peptide bond: a carbon electrophile (carbonyl) and a nucleophile (amine).

### Example 8

A second general way to make peptide multimers is the linking of peptide sequences with intervening linker moieties in a linear fashion, introduced above as the “serial” approach. Serial peptide multimers are thus assembled via the coupling of complementary functional groups attached to the ends of the peptides. Serial homing peptide multimers are expected to display different properties than homing peptide multimers constructed in a “parallel” fashion.

One end of a homing peptide is converted to a vinyl functional group via reaction with an allylic electrophile, e.g. allyl bromide. Such a vinyl group is then epoxidized using methods known to those skilled in the art of organic synthesis. The result of such a synthetic sequence is a homing peptide having an electrophillic epoxide functional group useful for coupling to a nucleophillic functional group on a second homing peptide, via a ring-opening addition shown in Equation 8. A product of such a ring-opening addition is shown in the bottom right hand side of Equation 8; this homing peptide “dimer” contains a new bond between the nucleophillic functional group of the first homing peptide and an electrophillic carbon of the functional group attached to a second homing peptide. Such linkages are stable to hydrolysis and co-generate an additional functional group (in this case a hydroxy group). Indeed, the bond forming reaction between homing peptides (the epoxide ring-opening step) may be conveniently carried out in water, the solvent of choice for peptide chemistry.

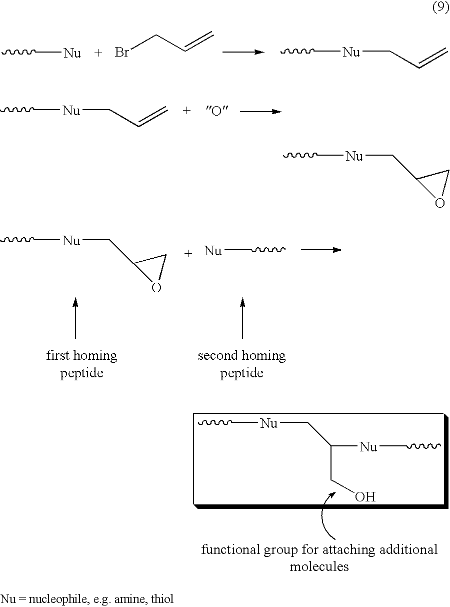

### Example 9

In a preferred aspect of the present invention, one end of a peptide chain is conjugated to a functional group comprising a carbon nucleophile, most preferably an organoboron or organostannane moiety as previously described above; the terminus of another peptide sequence is transformed into a complementary carbon electrophile, most preferably, into an alkyl-, vinyl-, aryl-, or acetylenic-halide, sulfonate, or acetate. Such complementary functional groups do not ordinary react at appreciable rates, but readily do so in the presence of a catalyst, for example, certain low valent transition metal complexes already describe above. One way to link to peptides makes use of the modified Suzuki reaction (Eq 10).

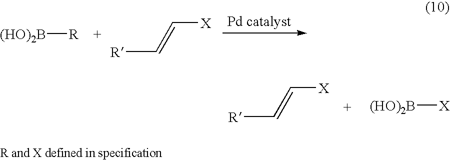

The expected product of the coupling of two peptides, one of which is functionalized with an organoboronate and the other with a vinyl halide, is a linked peptide “dimer” having a linker which retains an olefinic group. One highly preferred aspect of this invention is that when coupling a carbon nucleophile to an unsaturated electrophile for example a vinyl halide, the stereochemistry (cis vs trans or E vs Z) about olefinic bond is retained (Eqs 10 and 11); thus for example a carbon electrophile have a “trans” geometry will give a linker comprising an olefin having a “trans” geometry; likewise, a carbon electrophile have a “cis” geometry will give a linker comprising an olefin having a “cis” geometry (Eq 11). Methods of generating requisite cis or trans vinyl reagents are well known to those skilled in the art.

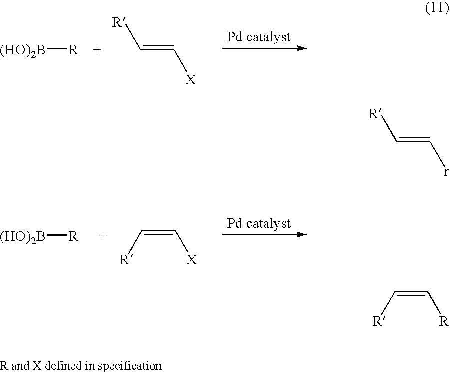

This common feature of the Stille, modified Suzuki, and related C—C coupling reactions is a preferred aspect of the present invention when viewed in the context of coupling peptides to make peptide multimers.

### Example 10

A highly preferred embodiment of the present invention is the interchangeability of complementary (electrophilic/nucleophilic) functional groups with respective to the different ends of peptides to be coupled. For example, one peptide is functionalized at either terminus (C or N) with either of the two complementary functional groups, FG₁ or FG₂, yielding four possible permutations (Eq 12).

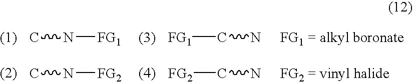

Thus the same homing peptide sequence may yield four distinct linkage combinations; each of these combinations in turn yields physically distinct homing peptide multimers, each of which may have different homing properties. In this preferred aspect of the present invention, the functionalized homing peptides are interchangeable building blocks for the selective construction of homing peptide multimers.

The unsaturated carbon-carbon bond is one of the most versatile function groups in organic chernistry. Further chemical elaboration of the newly formed olefinic functional group, wherein said olefinic functional group is present as a consequence of coupling an allcene carbon electrophile with an alkyl boronate, provides a convenient point of attachment for a drug, prodrug, or other therapeutic agent.

Selective oxidation or functionalization of the newly formed olefinic group e.g., the selective dihydroxylation or epoxidation of that olefinic bond, may improve or otherwise alter the solubility properties of the peptide multimer and introduces additional asymmetric centers which may fundamentally transform the physical properties of the homing peptide multimers. For example, hydrophilic or hydrophobic groups may be subsequently appended to the newly formed olefinic or unsaturated functional group. The degree of flexibility, the extent of hydration, and the size of the scaffold may play important roles in the design of homing peptide multimers. Countless elaborations of olefinic functional groups are well known to those of ordinary skill in the art and are included here by provident suggestion.

### Example 11

In another preferred aspect of the present invention, complementary functional groups are attached to both ends of homing peptide sequence and, in the absence of intramolecular coupling (which may be avoided or disfavored at high functionalized homing peptide concentrations) intermolecular coupling occurs and yields a functionalized homing peptide dimer containing a linker group (Eq 13).

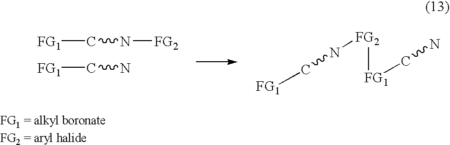

By analogy to monofunctionalized homing peptide sequences, controlled selective functionalization of the same or different homing peptides yields myriad functionalized homing peptide multimers, each of them physically distinguishable and each having different affinity properties for the intended cellular target. A monofunctionalized dimer may be coupled with yet another homing peptide having the complementary functional group to give a homing peptide “trimer” (Eq 14).

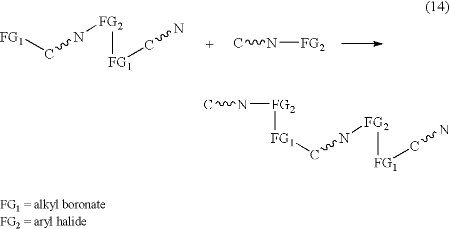

Depending on the coupling partners chosen, each of the linkers between the horning peptide contains points of unsaturation, enabling further elaboration and or attachment of other molecules, for example, drugs or prodrugs.

Suzuki couplings are known in the art to be water insensitive and Stille couplings are routinely carried out in polar organic solvents and the reaction is water tolerant. Thus another preferred aspect of the homing peptide coupling methodologies presented herein is their compatibility or tolerance of water and/or polar non aqueous solvents, e.g., DMSO, DMF, the solvents of choice for peptide chemists.

While reference has been made to particular preferred embodiments and to several uses and applications made possible by the invention, it will be understood that the present invention is not to be construed as limited to such, but rather to the lawful scope of the appended claims and subject matter covered by the doctrine of equivalents.

We claim:

1. A homing peptide multimer comprising a first homing peptide associated with a second homing peptide, wherein the first and second homing peptides comprise the same sequence of amino acid residues.

2. A homing peptide multimer according to claim 1 wherein the first homing peptide is associated with the second homing peptide through a linker.

3. A homing peptide multimer according to claim 2 wherein the first homing peptide is covalently linked to the second homing peptide through a linker.

4. A homing peptide multimer according to claim 3 wherein the linker comprises one or more amino acids.

5. A homing peptide multimer according to claim 4 wherein the linkage between the first homing peptide and the linker is a peptide bond.

6. A homing peptide multimer according to claim 4 wherein the linkage between the second homing peptide and the linker is a peptide bond.

7. A homing peptide multimer according to claim 4 wherein the linkages between the first homing peptide and the linker and the second homing peptide and the linker are peptide bonds.

8. A homing peptide multimer according to claim 1 further comprising at least one additional peptide.

9. A homing peptide multimer according to claim 8 wherein at least one of the additional peptides is another homing peptide.

10. A homing peptide multimer according to claim 9 wherein at least one additional homing peptide comprises the same sequence of amino acid residues as the first and second homing peptides.

11. A homing peptide multimer according to claim 9 wherein at least one additional homing peptide comprises a different sequence of amino acid residues than the first and second peptides.

12. A homing peptide multimer according to claim 8 wherein at least one of the additional peptides is a therapeutic agent.

13. A homing peptide multimer according to claims 1-12 that is a pharmaceutically acceptable salt.

14. A composition comprising a homing peptide multimer according to claim 13 and a carrier.

15. A composition according to claim 14 wherein the carrier is a pharmaceutically acceptable carrier.

16. A composition according to claim 14 or 15 that is a substantially dry composition.

17. A composition according to claim 14 or 15 that is a liquid composition.

18. A homing peptide multimer according to formula (1):
[HP₁]-[[L-HPₐ]]ₓ (1)
wherein “HP₁” is a first homing peptide that comprises an HP, amino acid sequence, “L” is a linker, “HPₐ” is a homing peptide, and “x” is an integer equal to at least 1, and when x is two or more, each HPₐ is independently selected, but at least one of the HPₐ homing peptides also comprises a HP₁ amino acid sequence.

19. A homing peptide multimer according to claim 20 wherein the homing peptides are covalently linked to the linker.

20. A homing peptide multimer according to claim 18 wherein the linker is covalently attached to the C- or N-terminus of HP₁, and is covalently attached to the C- or N-terminus of HPₐ.

21. A homing peptide multimer according to claim 18 wherein at least one of the homing peptides is a tumor homing peptide.

22. A homing peptide multimer according to claim 18 that is a pharmaceutically acceptable salt.

23. A composition comprising a homing peptide multimer according to claim 18 and a carrier.

24. A composition comprising a homing peptide multimer according to claim 18 and a pharmaceutically acceptable carrier or diluent.

25. A composition according to claim 24 that is a dry composition.

26. A composition according to claim 24 that is a liquid composition.

27. A method of making a peptide multimer comprising the steps of:
(a) providing a first peptide with one or more carbon electrophile or carbon nucleophile,
(b) providing a second peptide with one or more carbon electrophile or nucleophile, whichever is complimentary to the reactive moiety in the first peptide,
(c) linking the complementary carbon electrophiles or nucleophiles of said first and second peptides to form a peptide multimer comprising a linker and the first and second peptides.

28. A method of making a peptide multimer according to claim 27 wherein: additional peptides units are provided with complementary carbon electrophiles or nucleophiles and linked to a peptide multimer formed in steps (a)-(c) above, via the sequential coupling of complementary carbon electrophiles or nucleophiles.

29. A method according to claim 27 wherein a carbon electrophile or nucleophile is selectively attached to the C- or N-terminus of a first peptide.

30. A method according to claim 27 wherein a carbon electrophile or nucleophile is optionally attached to the C- or N-terminus of a second peptide.

31. A method according to claim 27 wherein the linker comprises a carbon-carbon or carbon-heteroatom bond not present before coupling.

32. A method according to claim 27 wherein one or more of the linked peptides is a tumor homing peptide.

33. A method according to claim 28 wherein said complementary carbon electrophiles or nucleophiles are optionally attached to the C- or N-terminus of ah additional peptide

34. A method according to claim 28 wherein at least two linked peptides comprise the same sequence of amino residues.

35. A method according to claim 27 wherein the linking step methodology (c) is a palladium catalyzed coupling methods selected from the group consisting of a modified Suzuki, Heck, Stille, or Sonagashira coupling.

36. A method according to claim 31 wherein said carbon-carbon bond is unsaturated

37. A method according to claim 36 wherein said unsaturated bond is formed with retention stereochemistry at the carbon electrophile.

38. A method of claim 36 wherein said carbon-carbon bond is subsequently selectively oxidized.

39. A method of claim 36 wherein said carbon-carbon bond is subsequently used as the point of attachment of a therapeutic agent or linker thereto.

40. A homing peptide multimer, wherein the homing peptide multimer comprises a scaffold molecule having a plurality of equivalent linkage moieties, one of which linkage moieties is linked to a first homing peptide and a second of which linkage moieties is linked to a second homing peptide, wherein the first and second homing peptides comprise the same sequence of amino acid residues.

41. A homing peptide multimer according to claim 40 wherein the scaffold molecule comprises a dendrimer comprising a plurality of equivalent termini, wherein at least two of such termini are independently coupled to a homing peptide.

42. A homing peptide multimer of the formula 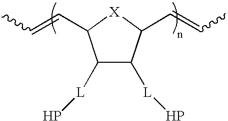
wherein X is O or CH₂,
wherein L is linker,
wherein HP is a homing peptide.

43. A homing peptide multimer according to claim 42 wherein “n” is less than 10.

44. A homing peptide multimer of claim 43 wherein each L is independently linked to a homing peptide, wherein each homing peptide comprises a homing peptide sequence.

45. A homing peptide multimer according to claim 44 wherein the homing peptide sequence of each homing peptide comprises the same sequence of amino acid residues.

46. A homing peptide multimer of claim 43 wherein n>2 and L is covalently linked to two or more different homing peptides.

47. A homing peptide multimer of claims 42-45 wherein n is greater than 10.

48. A method of synthesizing a homing peptide multimer comprising the steps of:
(1) attaching a homing peptide, B:P to strained olefin monomer via a linker, L.
(2) treating the product of step (1) with an olefin metathesis catalyst

49. A method of extending a homing peptide multimer prepared according to claim comprising the additional step of
(3) Treating the product of step (2) with additional olefin monomer which is covalently bonded to a different homing peptide than BY in step (1).

50. A method of delivering a therapeutic agent comprising contacting a cell wherein a therapeutic agent comprises a homing peptide multimer and a drug or prodrug.

51. A method according to claim 50 wherein the cell is in vivo.

52. A method according to claim 50 wherein the cell is in vitro.

53. A method according to claim 50 wherein said drug or a prodrug is covalently attached to the homing peptide multimer.

54. A method according to claim 50 wherein the therapeutic agent is a nucleic acid.

55. A method according to claim 50 wherein the therapeutic agent is a protein.

56. A method according to claim 50 wherein the therapeutic agent is a lipid.

57. A method according to claim 50 wherein the therapeutic agent is a carbohydrate.
